(19) United States
(12) Patent Application Publication
(10) Pub. No.: US 2006/0100596 A1
(43) Pub. Date: May 11, 2006

(54) Undergarment for a male incontinence device

(76) Inventor: Mark Miskie, Charlotte, NC (US)

Correspondence Address: Schwartz Law Firm, P.C.;SouthPark Towers, Suite 530, 6100 Fairview Road, Charlotte, NC, 28210, US

(21) Appl. No.: 10/983,500

(22) Filed: Nov. 8, 2004

(51) Int. Cl. A61F 5/44

(52) U.S. Cl. 604/349

(57) ABSTRACT

An undergarment is adapted for use in combination with a male incontinence device. The incontinence device includes an external fluid-transfer element for being positioned on a user inside of the undergarment. The fluid-transfer element has a transition portion adapted for extending outside of the undergarment to a fluid collection site. The undergarment includes a waistline, and a front and rear joined to the waistline and defining respective first and second leg openings. An enlarged, or expandable, crotch panel is located in the front, and includes a fabric gathering adapted for accommodating positioning of the fluid-transfer element on the user. A transition hole is formed at a base of the enlarged crotch panel, and is adapted for receiving the transition portion of the fluid-transfer element extending from the inside of the undergarment to the outside of the undergarment.

### TECHNICAL FIELD AND BACKGROUND OF THE INVENTION

This invention relates generally to male incontinence, and more specifically to an undergarment adapted for use with a male incontinence device. The invention supports the incontinence device securely and comfortably on the user without adhesives, tape, glue, or other constricting elements which may irritate and injure the skin. The invention is launderable and reusable, and provides a freedom of movement often sacrificed with other support systems for commercially available male incontinence devices and catheters. Users can wear the present invention with confidence and without a feeling of self-consciousness.

Incontinence is a growing problem, particularly in those adults ages 65 and older. Approximately two million males in the United States suffer moderate to severe incontinence. Common causes for this condition include an enlarged prostate, prostate cancer, pelvic trauma, spinal cord injury, and medication side-effects. Over $16 billion is spent annually on incontinence related care. Several devices used for managing male incontinence include a reusable fluid collection bag intended to reside beneath the outer clothing of the user. While these such devices typically offer increased collection capacity and are cleanable, the general lack of comfort, convenience, and confidence are drawbacks.

The present invention addresses these drawbacks. Specifically, the invention provides a unique suspension system which comfortably and securely holds the incontinence device in place during use. The invention promotes self-esteem and increased confidence by reducing the risk of accidents caused by disconnecting or improperly positioned parts. The undergarment closely resembles a conventional sport brief, and is easily put on and removed. Used in combination with the incontinence device, the invention offers a better quality of life for those active wearers suffering from moderate to heavy incontinence.

### SUMMARY OF THE INVENTION

Therefore, it is an object of the invention to provide an incontinence device support and positioning system in an undergarment especially adapted for a male incontinence device including an external fluid-transfer receptacle.

It is another object of the invention to provide an incontinence device support and positioning system which is non-irritating to the skin, and which does not use adhesives, tape, glue or other constricting elements attached directly to the penis.

It is another object of the invention to provide an incontinence device support and positioning system which is launderable and reusable.

It is another object of the invention to provide an incontinence device support and positioning system which resembles a conventional sport brief, and is easy to put on and remove.

It is another object of the invention to provide an incontinence device support and positioning system which is especially designed for active adults.

It is another object of the invention to provide an incontinence device support and positioning system which is designed for all day wear without changing.

It is another object of the invention to provide an incontinence device support and positioning system which offers increased comfort, confidence, and convenience over existing commercially available systems.

These and other objects of the present invention are achieved in the preferred embodiments disclosed below by providing an undergarment for use in combination with a male incontinence device. The incontinence device includes an external fluid-transfer element for positioning on a user inside of the undergarment. The fluid-transfer element has a transition portion adapted for extending outside of the undergarment to a fluid collection site. The undergarment includes a waistline, and a front and rear joined to the waistline and defining respective first and second leg openings. An enlarged, or expandable, crotch panel is located in the front, and includes a fabric gathering adapted for accommodating positioning of the fluid-transfer element on the user. A transition hole is formed at a base of the enlarged crotch panel, and is adapted for receiving the transition portion of the fluid-transfer element extending from the inside of the undergarment to the outside of the undergarment.

The “fluid-transfer element” may include any wholly or substantially external attachment, such as a condom-type catheter, or unattached rigid or semi-rigid covering or receptacle designed to fit over or within a portion of the penis. The “transition portion” of the fluid-transfer element is defined as any extension or passage from the fluid-transfer element inside of the undergarment to a collection site outside of the undergarment. The transition portion may be, for example, an elongated neck of the fluid-transfer element and/or a separate tube connected to the fluid-transfer element.

According to another preferred embodiment of the invention, the enlarged crotch panel has a fabric pleat formed adjacent to the transition hole at the base of the crotch panel. Alternatively, the gathering in the crotch panel may be formed by a dart or other suitable pouch-forming means.

According to another preferred embodiment of the invention, the fabric pleat extends laterally across the crotch panel.

According to another preferred embodiment of the invention, the front and rear cooperate to further define first and second leg segments which communicate with respective first and second leg openings.

According to another preferred embodiment of the invention, means are formed with one of the first and second leg segments for retaining a collection bag adapted for receiving and storing fluid discharged by the user through the fluid transfer element.

The term “collection bag” is defined broadly herein to include any rigid, semi-rigid, or flexible vessel suitable for holding fluid.

According to another preferred embodiment of the invention, the means for retaining includes a pocket formed with the leg segment on the outside of the undergarment.

Alternatively, the means for retaining may simply include complementary snaps located on the bag and undergarment, or complementary touch fasteners.

According to another preferred embodiment of the invention, the pocket includes a horizontal opening adapted for receiving the collection bag.

According to another preferred embodiment of the invention, the pocket includes a touch fastener adapted for mating with a complementary touch fastener located on the collection bag.

According to another preferred embodiment of the invention, an element retaining strap is located inside of the undergarment and above the transition hole to further position the fluid-transfer element inside the undergarment.

According to another preferred embodiment of the invention, a horizontal fly is formed with the front of the undergarment.

According to another preferred embodiment of the invention, the horizontal fly is located above the transition hole formed at the base of the crotch panel.

According to another preferred embodiment of the invention, the horizontal fly includes complementary snap fasteners for releasably closing the fly.

### BRIEF DESCRIPTION OF THE DRAWINGS

Some of the objects of the invention have been set forth above. Other objects and advantages of the invention will appear as the description proceeds when taken in conjunction with the following drawings, in which:

FIG. 1 is a perspective view of an undergarment according to one preferred embodiment of the present invention, and showing the male incontinence device in an in-use assembled condition positioned and supported by the undergarment;

FIG. 2 is a perspective view of the undergarment with the male incontinence device in a disassembled condition with elements exploded away for clarity;

FIG. 3 is a fragmentary perspective view of the undergarment showing the crotch panel and extension of the fluid-transfer receptacle through the transition hole;

FIG. 4 is a fragmentary cross-sectional view of the undergarment taken substantially at the transition hole of the crotch panel, and showing the positioning of the receptacle inside the undergarment;

FIG. 5 is a fragmentary perspective view of the undergarment showing the pleated area of the expandable crotch panel; and

FIG. 6 is a fragmentary cross-sectional view of the undergarment showing the pleat of the expandable crotch panel.

### DESCRIPTION OF THE PREFERRED EMBODIMENT AND BEST MODE

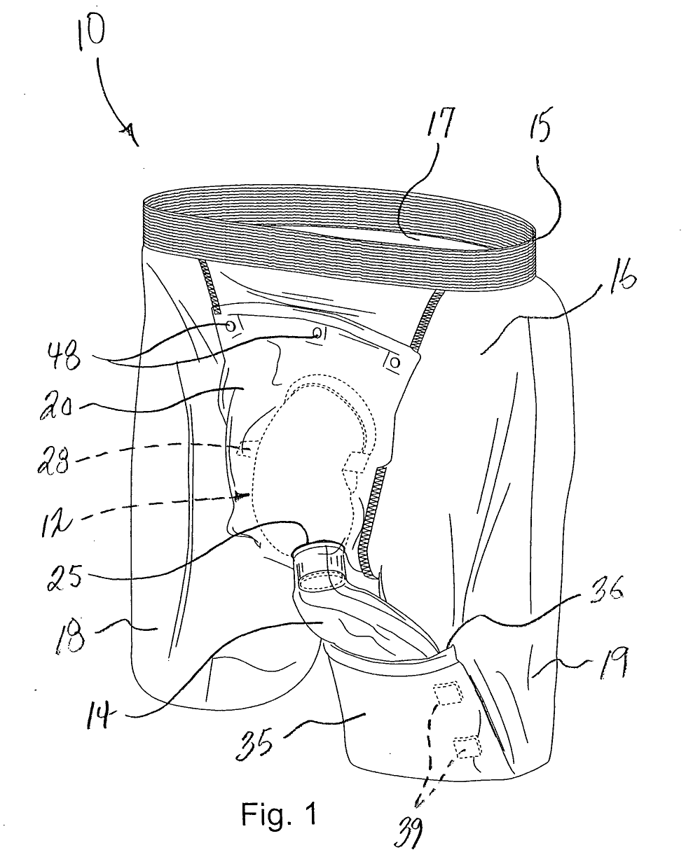

Referring now specifically to the drawings, an undergarment according to the present invention is illustrated in FIG. 1, and shown generally at reference numeral 10. The undergarment 10 is especially designed for use in combination with a male incontinence device 11. Although the undergarment 10 is usable with many different types of incontinence devices, the device 11 shown comprises an external fluid-transfer receptacle 12 and a collection bag 14. The undergarment 10 resembles a conventional sport brief including an elastic waistline 15, a front and rear 16 and 17 joined to the waistline 15, and first and second leg segments 18 and 19 defining respective leg openings. An enlarged crotch panel 20 is located in the front 16, and defines an expandable fabric gathering sufficient to accommodate positioning of the fluid-transfer receptacle 12 on the user. The undergarment 10 is preferably constructed of a soft knit fabric, such as cotton, and may include moisture-wicking fibers in an area of the crotch for managing incidental, minor wetting.

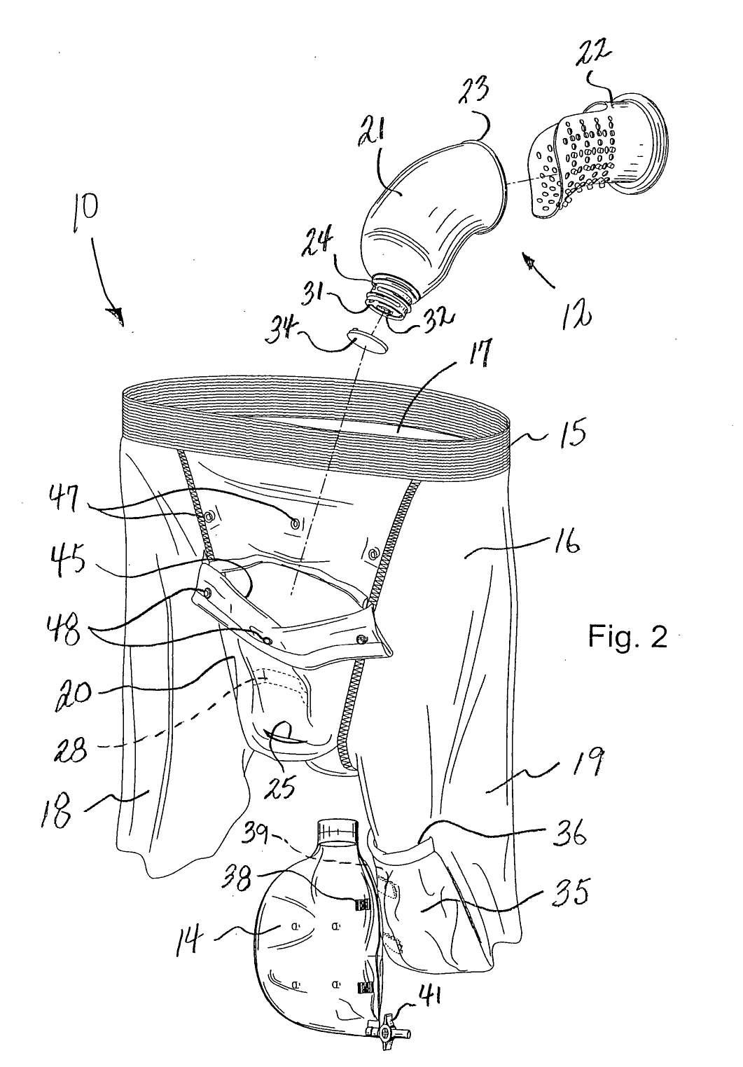

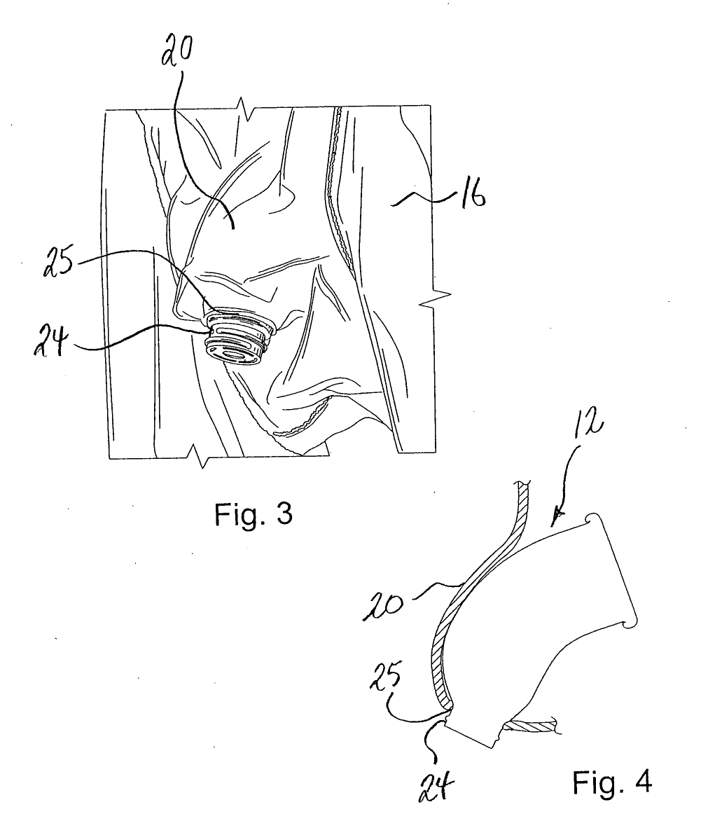

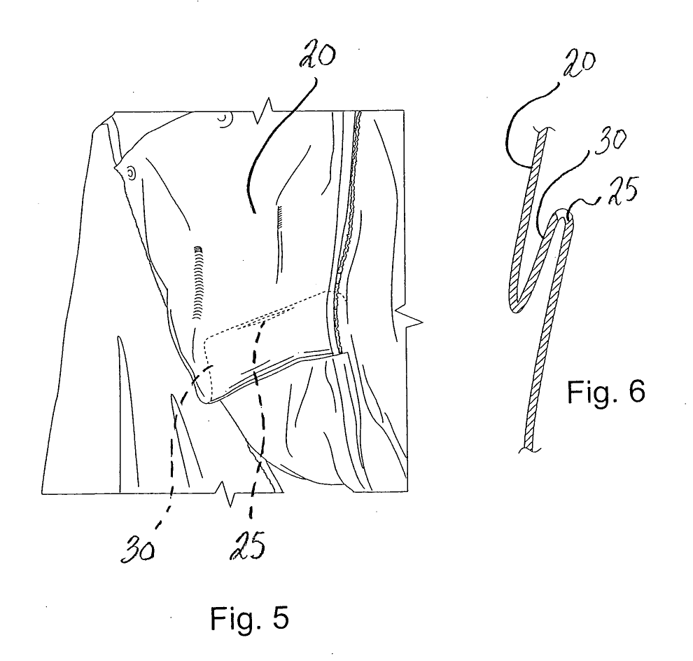

As best shown in FIG. 2, the fluid-transfer receptacle 12 includes an ergonomically-designed, shape-retaining outer shell 21 and detachable fluid-management insert 22. The outer shell 21 has a relatively large diameter open end 23 adapted for receiving the penis, and an opposite end defining an elongated reduced-diameter neck 24. The neck 24 defines a transition portion designed to penetrate the crotch panel 20 of the undergarment 10 through a transition hole 25, as shown in FIGS. 3 and 4. The transition hole 25 is formed at the base of the crotch panel 20, and cooperates with an elastic retaining strap 28 to further position the receptacle 12 inside the undergarment 10 during use. In one embodiment, the crotch panel 20 has a laterally-extending pleat 30, shown in FIGS. 5 and 6, which expands upon placement of the receptacle 12 over the penis. The receptacle 12 is maintained in a generally upright position when standing, and a generally nose-down position when seated to avoid substantial backflow of urine towards the user.

Referring again to FIGS. 1 and 2, an end wall 31 of the neck 24 defines a fluid discharge port 32 through which urine passes outwardly from the receptacle 12 and into the collection bag 14. A disk-shaped, one-way flap valve 34 located between the discharge port 32 and the collection bag 14 controls the flow of urine from the receptacle 12 to the bag 14. The collection bag 14 attaches to the threaded neck 24 at the discharge port 32 and fits within a leg pocket 35 formed with the undergarment 10. The pocket 35 has a horizontal opening 36, and is sufficiently roomy to loosely contain the bag 14 without applying unnecessary pressure on the bag 14. The pocket 35 further serves to position the bag 14 adjacent the thigh of the user to prevent accidental compression caused by sitting on the bag or squeezing the bag between the legs. Preferably, the collection bag 14 is releasably retained inside the pocket 35 using complementary patches 38 and 39 of hook and loop touch fasteners. When the collection bag 14 is full, urine is drained through an outlet valve 41.

For added convenience, the user may choose to urinate outside of the receptacle 12 in a “normal” manner through a horizontal fly 45 formed in the front 16 of the undergarment 10. The horizontal fly 45 is located along a top edge of the crotch panel 20, and is releasably closed using complementary snap fasteners 47 and 48.

An undergarment for use in combination with a male incontinence device is described above. Various details of the invention may be changed without departing from its scope. Furthermore, the foregoing description of the preferred embodiment of the invention and best mode for practicing the invention are provided for the purpose of illustration only and not for the purpose of limitation—the invention being defined by the claims.

I claim:

1. An undergarment for use in combination with a male incontinence device, the incontinence device including an external fluid-transfer element for being positioned on a user inside of said undergarment and having a transition portion thereof adapted for extending outside of said undergarment to a fluid collection site, said undergarment comprising:
(a) a waistline, and a front and rear joined to the waistline and defining respective first and second leg openings;
(b) an enlarged crotch panel located in said front and comprising a fabric gathering adapted for accommodating positioning of the fluid-transfer element on the user; and
(c) a transition hole formed at a base of said enlarged crotch panel, and adapted for receiving the transition portion of the fluid-transfer element extending from the inside of said undergarment to the outside of said undergarment.

2. An undergarment according to claim 1, wherein said enlarged crotch panel comprises a fabric pleat formed adjacent to said transition hole at the base of said crotch panel.

3. An undergarment according to claim 2, wherein said fabric pleat extends laterally across said crotch panel.

4. An undergarment according to claim 1, wherein said front and rear cooperate to further define first and second leg segments communicating with respective first and second leg openings.

5. An undergarment according to claim 1, and comprising means formed with one of said first and second leg segments for retaining a collection bag adapted for receiving and storing fluid discharged by the user through the fluid-transfer element.

6. An undergarment according to claim 5, wherein said means for retaining comprises a pocket formed with said leg segment on the outside of said undergarment.

7. An undergarment according to claim 6, wherein said pocket includes a horizontal opening adapted for receiving the collection bag.

8. An undergarment according to claim 7, wherein said pocket comprises a touch fastener adapted for mating with a complementary touch fastener located on the collection bag.

9. An undergarment according to claim 1, and comprising an element retaining strap located inside of said undergarment and above said transition hole to further position the fluid-transfer element inside said undergarment.

10. An undergarment according to claim 1, and comprising a horizontal fly formed with the front of said undergarment.

11. An undergarment according to claim 10, wherein said horizontal fly is located above said transition hole formed at the base of said crotch panel.

12. An undergarment according to claim 10, wherein said horizontal fly comprises complementary snap fasteners for releasably closing said fly.

13. In combination with a male incontinence device comprising an external fluid-transfer element for being positioned on a user and a collection bag for receiving and storing fluid discharged by the user through the fluid-transfer element, an undergarment comprising:
(a) a waistline, and a front and rear joined to the waistline and defining respective first and second leg openings;
(b) an enlarged crotch panel located in said front and comprising a fabric gathering adapted for accommodating positioning of the fluid-transfer element on the user inside of said undergarment; and
(c) a transition hole formed at a base of said enlarged crotch panel, and adapted for receiving a transition portion of the fluid-transfer element extending from the inside of said undergarment to the collection bag located outside of said undergarment.

14. A combination according to claim 13, wherein said enlarged crotch panel comprises a fabric pleat formed adjacent to said transition hole at the base of said crotch panel.

15. A combination according to claim 14, wherein said fabric pleat extends laterally across said crotch panel.

16. A combination according to claim 13, wherein said front and rear cooperate to further define first and second leg segments communicating with respective first and second leg openings.

17. A combination according to claim 16, and comprising means formed with one of said first and second leg segments for retaining the collection bag.

18. A combination according to claim 17, wherein said means for retaining comprises a pocket formed with said leg segment on the outside of said undergarment.

19. A combination according to claim 18, wherein said pocket includes a horizontal opening adapted for receiving the collection bag.

20. A combination according to claim 19, wherein said pocket comprises a touch fastener adapted for mating with a complementary touch fastener located on the collection bag.
